SEQUENCE LISTING (1 sequence)

SEQ ID NO: 1 - DNA, 22 bp, Artificial sequence
  Synthetic
tgactgtgaacgttcgagatga

(19) United States
(12) Patent Application Publication
(10) Pub. No.: US 2023/0083448 A1
(43) Pub. Date: Mar. 16, 2023

(54) COMPOSITIONS OF ALUM NANOPARTICLES FOR IMMUNOMODULATION AND METHODS FOR PRODUCING THE SAME

(71) Applicant: The Johns Hopkins University, Baltimore, MD (US)

(72) Inventors: Hai-Quan Mao, Baltimore, MD (US); Gregory P. Howard, Baltimore, MD (US)

(21) Appl. No.: 17/798,064

(22) PCT Filed: Feb. 5, 2021
(86) PCT No.: PCT/US2021/016852

§ 371 Date: Aug. 5, 2022

Related U.S. Application Data
(60) Provisional application No. 62/971,674, filed on Feb. 7, 2020.

(51) Int. Cl. A61K 39/00; C01F 7/746; A61K 9/51; A61K 39/39; A61P 35/00; A61K 39/07; A61K 39/05; A61K 39/08; A61K 39/29; A61K 39/015; A61K 39/13; A61K 39/09; A61K 39/02; A61K 39/12

(52) U.S. Cl. CPC A61K 39/0011; C01F 7/746; A61K 9/5192; A61K 39/39; A61K 9/5169; A61K 9/5161; A61P 35/00; A61K 39/07; A61K 39/05; A61K 39/08; A61K 39/292; A61K 39/015; A61K 39/13; A61K 39/092; A61K 39/099; A61K 39/12; A61K 39/29; C01P 2004/64; C01P 2004/62; C01P 2004/04; A61K 2039/55505; A61K 2039/57; A61K 2039/55561; A61K 2039/55555

(57) ABSTRACT

An aluminum nanoparticle adjuvant carrier system with stabilizing surface coatings that can efficiently deliver protein or nucleic acid antigen payloads to naive, resident APCs is disclosed.

### BACKGROUND

Vaccination is responsible for the eradication of smallpox and the rapid decline of many infectious diseases. To maximize safety and minimize reactogenicity, vaccine development is gradually shifting from whole, inactivated vaccines to well-defined subunit vaccines. This approach, however, often suffers from reduced immunogenicity. Development of adjuvants that are safe and potentiate immune response and long-term immune memory is of paramount importance.

### SUMMARY

In some aspects, the presently disclosed subject matter provides a nanoparticle comprising an alum core and a coating, wherein the nanoparticle has a number average size between about 20 nm and about 300 nm and a polydispersity index between about 0.1 to about 0.3.

In some aspects, the alum core comprises an aluminum compound selected from the group consisting of aluminum hydroxide, aluminum phosphate, aluminum chloride, amorphous aluminum hydroxyphosphatesulfate (AAHS), potassium aluminum sulfate, and combinations thereof.

In some aspects, the surface coating is selected from the group consisting of one or more anionic polysaccharides, one or more cationic polymers, and one or more anionic polymers.

In certain aspects, the one or more anionic polysaccharides is selected from the group consisting of hyaluronic acid, heparin sulfate, chondroitin sulfate, and dextran sulfate.

In certain aspects, the one or more cationic polymers are selected from the group consisting of branched or linear polyethylenimine, poly(L-lysine), poly(P-amino esters), protamine, chitosan, and combinations thereof. In certain aspects, the one or more anionic polymers comprise cytosine phosphoguanosine (CpG) oligodeoxynucleotide.

In some aspects, the surface coating is crosslinked. In certain aspects, the crosslinked surface coating comprises thiolated hyaluronic acid, thiolated dextran sulfate, or nucleic acids modified with crosslinkable groups.

In some aspects, the nanoparticle further comprises a protein or peptide antigen entrapped within the coating. In other aspects, the nanoparticle further comprising a protein or peptide antigen conjugated to a surface of the coating.

In some aspects, the nanoparticle has a number average size between about 20 nm and about 200 nm.

In other aspects, the presently disclosed subject matter provides a vaccine adjuvant comprising one or more of the presently disclosed nanoparticles.

In other aspects, the presently disclosed subject matter provides a vaccine comprising one or more presently disclosed nanoparticles or a vaccine adjuvant thereof.

In some aspects, the vaccine further comprises one or more of a vaccine selected from the group consisting of Anthrax, DT, DTaP (Daptacel), DTaP (Infanrix), DTaP-IPV (Kinrix), DTaP-IPV (Quadracel), DTaP-HepB-IPV (Pediarix), DTaP-IPV/Hib (Pentacel), HepA (Havrix), HepA (Vaqta), HepB (Engerix-B), HepB (Recombivax), HepA/HepB (Twinrix), HIB (PedvaxHIB), HPV (Gardasil9), Japanese encephalitis (Ixiaro), MenB (Bexsero, Trumenba), Pneumococcal (Prevnar13), Td (Tenivac), Td (MassBiologics), Tdap (Adacel), Tdap (Boostrix), and malaria (RTS.S (Mosquirix)).

In certain aspects, the vaccine is a cancer vaccine. In particular aspects, the cancer vaccine is selected from the group consisting of BiovaxID (follicular lymphoma, a type of non-Hodgkin's lymphoma), sipuleucel-T (prostate cancer), oncophage (kidney cancer), and talimogene laherparepvec (melanoma), or a patient-derived neoantigen.

In other aspects, the presently disclosed subject matter provides a method for treating a disease or condition, the method comprising administering one or more presently disclosed nanoparticles to a subject in need of treatment thereof.

In some aspects, the treating is prophylactic. In other aspects, the treating is therapeutic.

In certain aspects, the nanoparticle drains to one or more lymph nodes.

In some aspects, the nanoparticle induces an anti-tumor response.

In some aspects, the administering of the nanoparticle is selected from the group consisting of intradermal (i.d.), subcutaneous (s.c.), and intramuscular (i.m.).

In other aspects, the presently disclosed subject matter provides for the use of the presently disclosed vaccine for treating or preventing an infectious disease, a cancer, and/or one or more other targets requiring cellular immunity for immunological protection. In certain aspects, the use is prophylactic or therapeutic.

In other aspects, the presently disclosed subject matter provides a method for preparing an alum nanoparticle, the method comprising admixing alum with a protein in a flash nanocomplexation apparatus.

In certain aspects, the method further comprises admixing a surface coating with the alum and protein in a flash nanocomplexation apparatus.

In particular aspects, the method comprises a one-step flash nanocomplexation process or a two-step flash nanocomplexation process.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 shows that, in the clinic, aluminum salt adjuvants form 1-μm to 20μm aggregates with antigen after bulk mixing, Shirodkar et al., 1990 (prior art);

FIG. 2A and FIG. 2B show that alum NPs can be generated using either: (FIG. 2A) a one- or (FIG. 2B) two-step FNC process;

FIG. 2C is a diagram showing that the presently disclosed alum NPs can be characterized in vitro using DCs and other innate immune cells to isolate formulations that skew selectivity toward Th1 response as determined by cytokine milieu after stimulation with NPs;

FIG. 3A, FIG. 3B, FIG. 3C, and FIG. 3D show that aluminum complexes with surface coatings are uniform as shown by TEM (FIG. 3A) and retain major chemical composition of constituent parts as demonstrated by FTIR (FIG. 3B). The Alum:OVA complexes with surface coatings without crosslinking are not stable as demonstrated by stabilized Alum:OVA nanoparticles over 2 h in buffered media with or without DTT to uncrosslink surface coatings;

FIG. 4A and FIG. 4B show uptake of OVA-FITC within (FIG. 4A) OVA:Al hydrogel complexes or; (FIG. 4B) stabilized Alum:OVA nanoparticles after 3 h of feeding. Scalebar=10 μm;

FIG. 5 shows Z-stack images of DCs treated with nothing (negative), free ovalbumin (OVA),OVA:Al hydrogel (OA), or stabilized Alum:OVA complexes were analyzed for colocalization of OVA in EEA1 (early endosome) and LAMP (lysosome) compartments to demonstrate that stabilized Alum:OVA complexes facilitate endosomal escape and localization in the cell cytosol;

FIG. 6 shows BALBc/J mice administered with stabilized Alum:OVA NPs show uptake in proximal iliac and distal axial lymph nodes after 3 h, showing passive targeting of these to the major draining lymph nodes without active cellular transport;

FIG. 7A, FIG. 7B, FIG. 7C, and FIG. 7D show stabilized aluminum nanoparticles with crosslinked coatings allowed for enhanced retention and penetration into both proximal and distal lymph nodes after s.c. administration. In the proximal iliac (FIG. 7A) and distal axial (FIG. 7C) lymph nodes, nanoparticles are seen penetrating into the paracortex region [insets] (FIG. 7B), (FIG. 7D). Scalebar=50 μm;

FIG. 8A and FIG. 8B illustrate a representative FNC HA-SH/Alum:OV NP preparation. FIG. 8A is screening of alum concentration with fixed 250 μg/mL OVA to determine optimal conditions for small Alum:OVA complexes. FIG. 8B is screening to determine optimal HA-SH (17.2% thiolation) concentration to stabilize NPs with low PDI (<0.3);

FIG. 9A, FIG. 9B, and FIG. 9C shows that NP size is a key determinate for (FIG. 9A, FIG. 9B) lymph node drainage and (FIG. 9C) retention in a PLGA-b-PEG NP model. ns p>0.05, *p<0.05, **p<0.01, ***p<0.001, n=6-10 mice per time point (prior art);

FIG. 10A, FIG. 10B, and FIG. 10C shows that modifying NP surface chemistry to include varying degrees of hydrophobicity has previously been demonstrated to impact transfection efficiency. A similar effect for immunogenicity is anticipated. (FIG. 10A) Linear poly(ethyleneimine) modified NPs with hydroxyl or methyl terminated functional groups of varying hydrophobicity (prior art). (FIG. 10B) Resulting transfection efficacy by functional group modification and grafting density. (FIG. 10C) HA-SH/Alum:OVA NPs surface functionalized using free surface thiols after NP crosslinking;

FIG. 11A and FIG. 11B show screening of AlCl₃.6H₂O with ovalbumin for generation of Alum:OVA complexes. (FIG. 11A) Various concentrations of AlCl₃.6H₂O dissolved in pH 2.00 water were screened with 250 μg/mL OVA in a 2-inlet flash nanocomplexation (FNC) device at a 10 mL/min flow rate. Nanoparticle hydrodynamic diameter, polydispersity (PDI), and (FIG. 11B) zeta potential was measured by dynamic light scattering. (FIG. 11B) The encapsulation efficiency (EE %) was determined by ultrafiltration of Alum:OVA complex followed by microBCA protein assay of filtrate;

FIG. 12A, FIG. 12B, FIG. 12C, FIG. 12D, FIG. 12E, and FIG. 12F show screening of thiolated and unthiolated hyaluronic acid (HA) coatings on Alum:OVA complexes. (FIG. 12A) A three-inlet FNC device was used, with one inlet have the 250-μg/mL OVA (25 mM HEPES, pH 11.00), the second with 0.5- or 1.0-mg/mL AlCl₃.6H₂O (deionized water, pH 2.0), and third inlet with 1-4 mg/mL (deionized, distilled water) pristine or thiolated HA. A 10 mL/min three-inlet FNC showed that (FIG. 12A) 35-kDa HA coating produced uniform nanoparticles all above 100 nm with (FIG. 12B) high encapsulation efficiency (EE) until excess 35-kDa HA coating was used. (FIG. 12C) Selecting the 1-mg/mL 35-kDa HA which gave the best EE and small size, higher flow rates of the three-inlet FNC were screened and it was found that sizes below 100-nm could not be obtained with a maximum flow rate. (FIG. 12D) On the other hand, using a 20-mL/min flow rate and lower molecular weight (4.7 kDa) thiolated HA (HA-SH, 15-20% substitution) yielded much smaller nanoparticles with (FIG. 12E) increasingly negative charge and with increasing HA-SH concentration. (FIG. 12F) Using the optimal small size condition of 1-mg/mL HA or HA-SH, it is evident that thiolated HA (15-20%) is necessary to obtain a small size below 100-nm for lymph node targeting;

FIG. 13A and FIG. 13B show the rate of nanoparticle crosslinking by aeration and gentle shaking. Two separate formulations (e.g., Formulation A, B) were selected from the previous screening and the degree of crosslinking was tested using a modified Ellman's reagent assay. (FIG. 13A) The crosslinking of nanoparticle plateaued after 48 h with some continued trend upward. (FIG. 13B) The quenching of nanoparticle surface thiols plateaued after 48 h with little consumption thereafter. Therefore, due to the abundance of available surface thiols in the first 48 h, thiolated ligands, antibodies, and other compounds can be conjugated onto the nanoparticle surface by just mixing the conjugate of interest with the nanoparticle mixture during crosslinking;

FIG. 14A, FIG. 14B, and FIG. 14C show the size control of HA-SH coated Alum:OVA complexes. Nanoparticles of different sizes were fabricated by modulating the flow rate of the inlet components to generate 4.7-kDa HA-SH coated

Alum:OVA complex nanoparticles. Nanoparticles were then crosslinked for 24 h by shaking and aeration. The size distribution of nanoparticles (FIG. 14A) fresh off the device and (FIG. 14B) after 24 h of crosslinking is shown. The (FIG. 14C) average size and polydispersity index (PDI) are together plotted as fresh off device (bold line) and after 24 h crosslinking (dotted line) at room temperature and shaking. This experiment demonstrates that nanoparticle size was mostly conserved during the crosslinking process with little change to size or PDI;

FIG. 15 demonstrates the necessity of each component for formation of small nanoparticles. The necessity of each component for formation of small HA-SH coated nanoparticles was demonstrated using a component subtraction study. Keeping all other components in the system same except for removing just AlCl₃.6H₂O, OVA, HA coating, or thiol groups compared to all components intact in the same was conducted. Only the HA-SH/Alum:OVA combination gave small (40 nm to 50 nm) size nanoparticle while subtracting thiolation gave large NP, no AlCl₃.6H₂O gave largest nanoparticles, and no HA coating gave unstable complexes. This experiment demonstrates the necessity of each component to yield small nanoparticles that could drain to the draining lymph node after s.c. or i.d. administration;

FIG. 16 shows a flow sorting strategy for measuring nanoparticle uptake. The above flow cytometry gating strategy was used to gate singlet murine immortalized dendritic cells (DC2.4) fed with nanoparticle treatments or control groups in vitro, and to measure corresponding uptake by FITC labeled OVA;

FIG. 17A, FIG. 17B, FIG. 17C, and FIG. 17D show the uptake of HA-SH NPs with various surface modifications by dendritic cell line (DC2.4). (FIG. 17A) The 40-nm HA-SH NP gave the best uptake compared to larger 60- and 120-nm HA-SH NPs after 3 h of uptake. (FIG. 17B) DC2.4 cells were fed with 25-100 μg/mL 35-kDa HA for 1 h and then given 40-nm HA-SH nanoparticles, free OVA, or no treatment for 3 h. It was shown that the 35-kDa HA blocking of CD44 receptor did not affect nanoparticle uptake, suggesting that these nanoparticles are taken up by another process other than CD44 mediated endocytosis. (FIG. 17C) The surface thiols on 40-nm HA-SH NP were modified with varying degrees of thiolated tri-mannose (modified 4-Aminophenyl1,3-α-1,6-α-D-mannotrioside, Synthose) conjugation or totally quenched using 2-mercaptoethanol and uptake observed after 3 h. (FIG. 17D) Varying OVA doses of HA-SH NP were added to each well and dose-dependent response observed. MFI is median fluorescence intensity;

FIG. 18A and FIG. 18B show T-cell stimulation by HA-SH NPs in splenocyte pool derived from C57BL6/J mice. (FIG. 18A) The percentage of OVA-specific CD8 T-cells after 7 days of splenocyte stimulation by nanoparticle treatments including Alum:OVA created by two inlet FNC, HA-SH NP freshly prepared without 24 h crosslinking (HA-SH NP −C), HA-SH NP crosslinked over 24 h (HA-SH NP +C), and positive control artificial antigen presenting cells (aAPCs) as previously published by Hickey et al. Nano Letters 2017. (FIG. 18B) Total count of splenocytes after 7 day treatment to show relative expansion of T-cells from each treatment;

FIG. 19A, FIG. 19B, FIG. 19C, FIG. 19D, FIG. 19E, and FIG. 19F show vaccination of C57BL6/J mice using NP treatments containing OVA antigen. Mice were vaccinated at days 0 and 14 with 10 μg endotoxin-free OVA and antibody collected by submandibular bleeding weekly. Groups included free OVA (OVA), OVA complexed with Alhydrogel (OVA:Alh), FNC produced Alum:OVA complexes without coating (FNC), 40-nm crosslinked HA-SH NPs (40 nm [+]), 40-nm HA-SH NPs without crosslinking (40-nm [−C]), and 100-nm crosslinked HA-SH NPs (100-nm [+]). (FIG. 19A-FIG. 19C) The half maximal effective concentration (EC50) of elicited OVA-specific polyclonal antibody from each treatment (10 μg) as measured by ELISA. (FIG. 19D-FIG. 19F) The total IgG antibody titer was calculated from the half-log serially diluted (from 500× to 1.58×10⁸×) mouse sera, and then plotted as an antibody kinetic over 56 days; and

FIG. 20A, FIG. 20B, FIG. 20C, and FIG. 20D show screening of adjuvant CpG 1018 ISS coating of Alum:OVA complexes using 3-inlet FNC. (FIG. 10A) Using 20-mL/min flow rate, various concentrations of CpG 1018 ISS were screened and corresponding nanoparticle size, potential, PDI, and (FIG. 20B) encapsulation efficiency (EE %) was measured. (FIG. 20C) Tuning the formulation and flow rate allowed for fine-tune size-control of dual adjuvant CpG 1018 ISS coated Alum:OVA nanoparticles. (FIG. 20D) A 3-h uptake assay in DC2.4 cells with FITC-OVA demonstrates that CpG coated NP had enhanced uptake relative to free FITC-OVA, albeit not as efficient as OVA Alhydrogel or HA/HA-SH coated complexes.

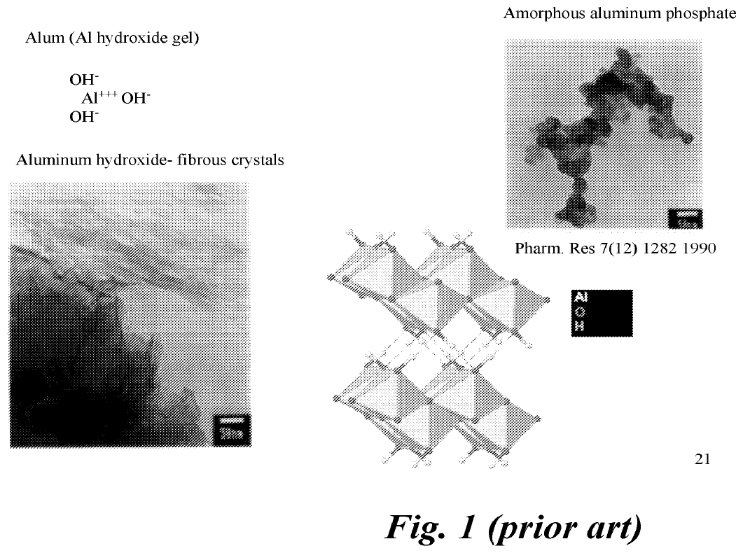

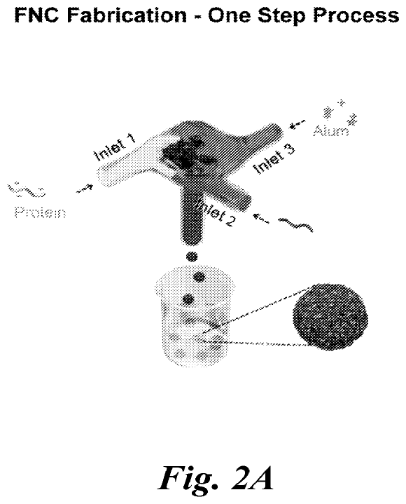

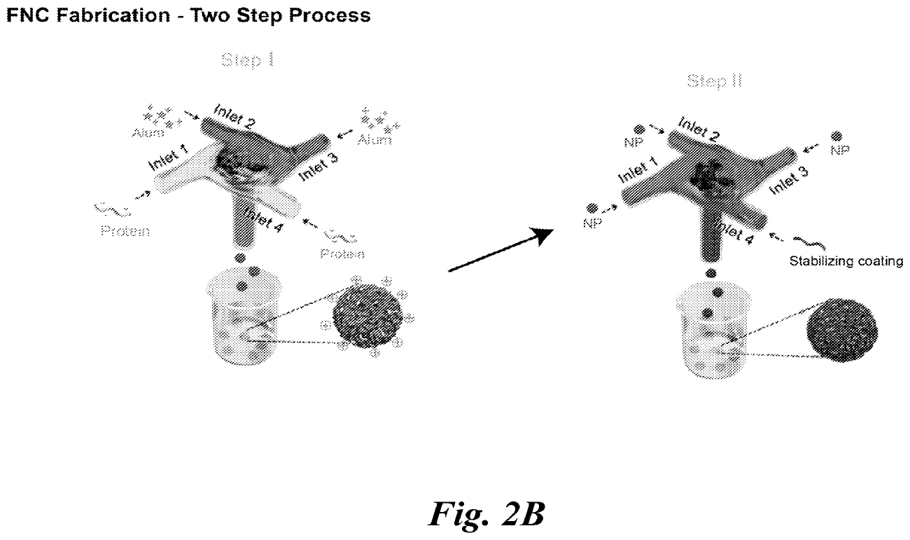

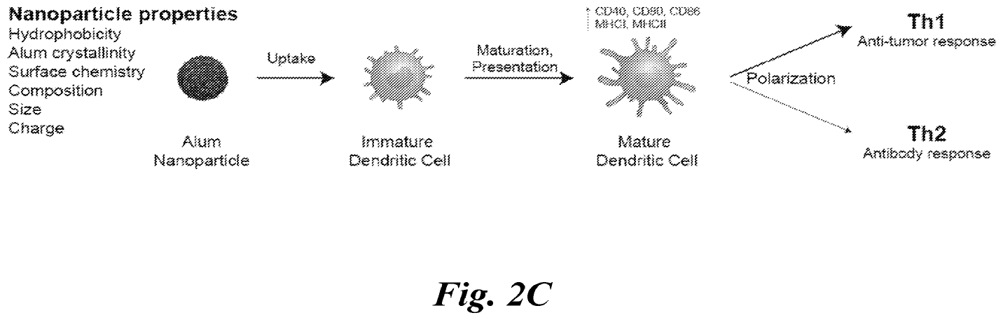

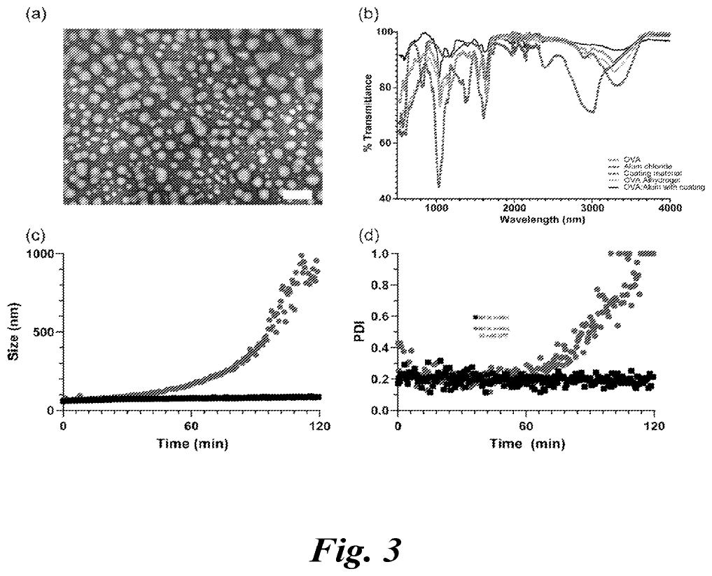

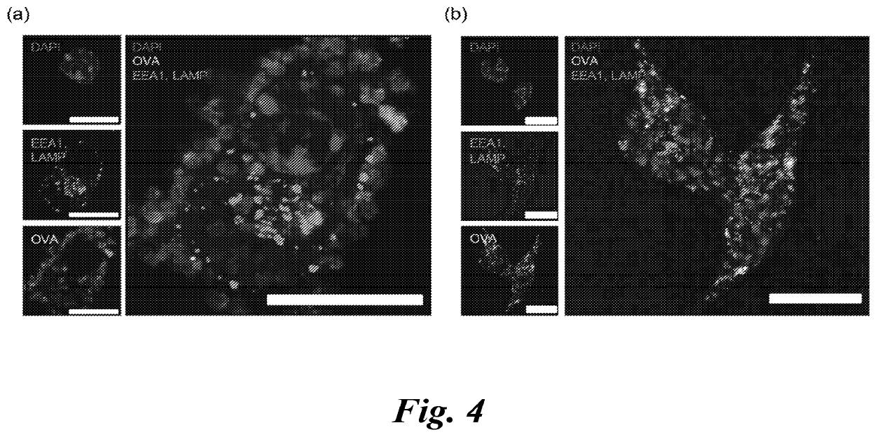

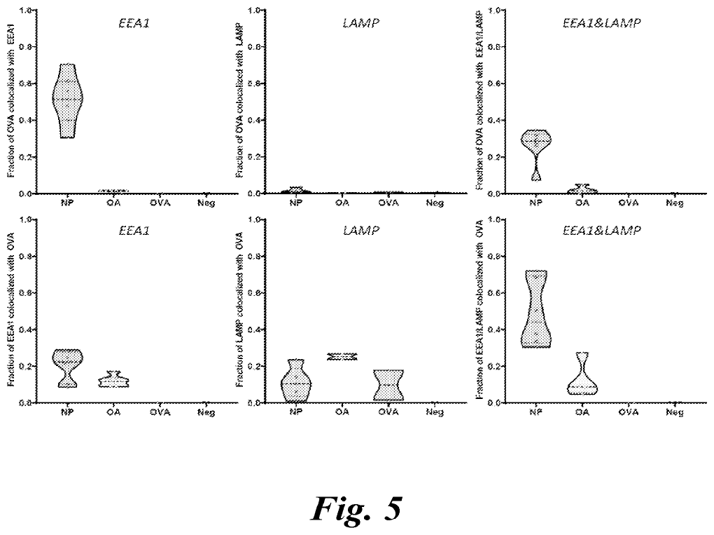

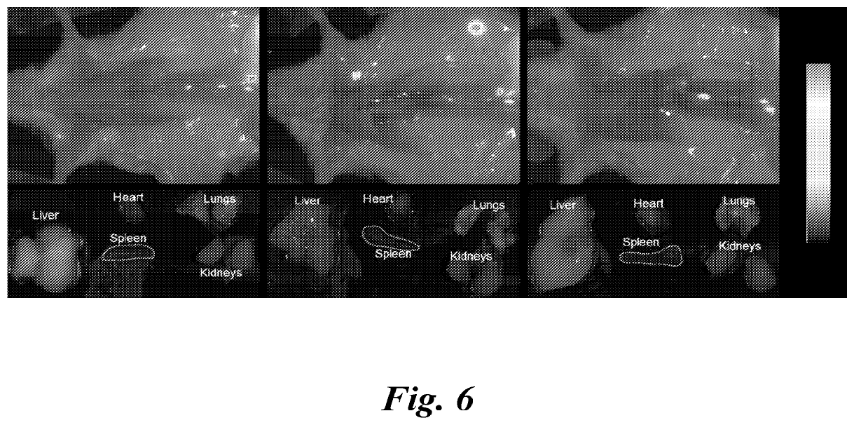

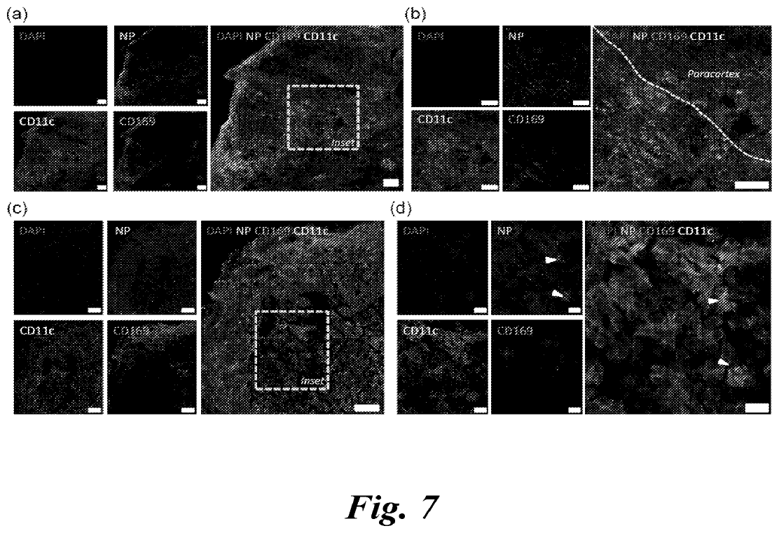

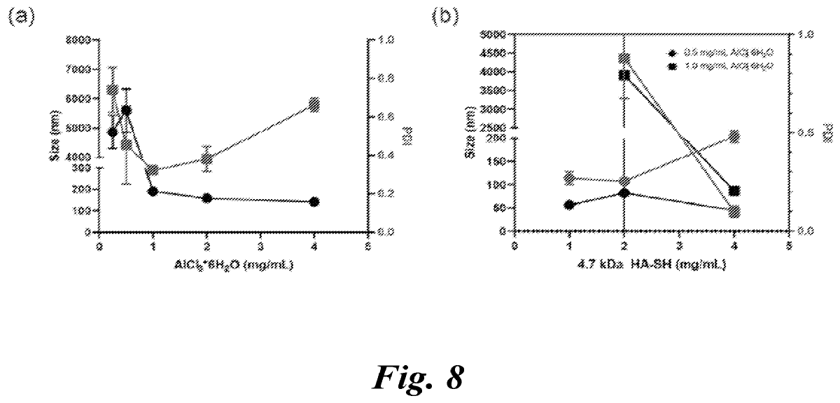

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Compositions of Alum Nanoparticles for Immunomodulation and Methods for Producing the Same

The presently disclosed subject matter provides a flash nanocomplexation (FNC) platform for producing small, uniform alum nanoparticles (NPs), which encapsulate protein or peptide antigens in a scalable and reproducible manner. The presently disclosed system allows for subsequent modifications of the alum NP including coatings and surface modifications. This characteristic affords a higher degree of control, allowing for the first thorough study of alum NP size and composition on Th1/Th2 immune response polarization. Combined with the safety record and successful history of FDA-approved alum adjuvants, the presently disclosed platform addresses a significant need in the immunotherapy field for Th1 adjuvants with high potential for clinical translation.

Aluminum salts (e.g., alum) were approved in 1934 for human use and currently are the most widely used adjuvants. Aluminum salt adjuvant include, but are not limited to, amorphous aluminum hydroxyphosphatesulfate (AAHS), aluminum hydroxide, aluminum phosphate, and potassium aluminum sulfate (Alum).

Aluminum salts are currently used as an adjuvant in the following approved vaccines: Anthrax, DT, DTaP (Daptacel), DTaP (Infanrix), DTaP-IPV (Kinrix), DTaP-IPV (Quadracel), DTaP-HepB-IPV (Pediarix), DTaP-IPV/Hib (Pentacel), HepA (Havrix), HepA (Vaqta), HepB (Engerix-B), HepB (Recombivax), HepA/HepB (Twinrix), HIB (PedvaxHIB), HPV (Gardasil9), Japanese encephalitis (Ixiaro), MenB (Bexsero,Trumenba), Pneumococcal (Prevnar13), Td (Tenivac), Td (MassBiologics), Tdap (Adacel), Tdap (Boostrix), and malaria (RTS.S (Mosquirix)). Aluminum salt adjuvants, however, lack utility for vaccine targets requiring cellular immunity as the mechanistic correlate of protection due to poor stimulation of CD8+T− cells and requirement of multiple boost doses for immune memory. Because of a focus on infectious disease vaccination, however, all adjuvants approved for human use are Th2-inducing. Accordingly, there is a clinical need for clinical need for Th1 adjuvants.

Recent studies demonstrate that alum salts fabricated as 100-200 nm nanoparticles (NPs) with surface-adsorbed antigen induce cellular immunity. Jiang et al., 2018; Wang et al., 2015. Aggregation after antigen adsorption, however, limits the feasibility to study size-dependent effects of these alum NPs. Without wishing to be bound to any one particular theory, it is thought that alum NP with well-defined and smaller size could yield higher efficiency of delivery to a rich population of immature dendritic cells (DCs) within the draining lymph nodes (LNs) via intradermal (i.d.) or subcutaneous (s.c.) administration. Wilson et al., 2003. Such a platform for producing uniform and small alum NPs that allow for skewing immune stimulation toward a Th1 response, however, is not available.

The presently disclosed subject matter aims to generate alum NPs with small (<100 nm) and controlled size, test their effectiveness to traffic to the draining LNs, assess its potency as an adjuvant to elicit strong Th1 immune response via antigen cross presentation.

The presently disclosed NPs exhibit superior dendritic cell activation relative to industry aluminum adjuvant controls and have promise as a Th1 adjuvant. Accordingly, the presently disclosed aluminum adjuvant NPs can be used to induce cellular immunity, thereby broadening the applicability of this approved adjuvant to cancer and other indications that still need vaccine adjuvants.

More particularly, the presently disclosed subject matter provides a reprogrammed alum adjuvant that generates cellular immunity. This alum adjuvant can be broadly applied to numerous targets including, but not limited to, cancer and infectious disease. Besides cytosine phosphoguanosine (CpG), there is a lack of approved adjuvants for cellular immunity induction. The presently disclosed alum adjuvant has the potential to be a ground-breaking improvement on current aluminum adjuvants since standard aluminum salts induce poor immune memory and little cellular immunity. In contrast, by controlling and stabilizing aluminum salts in nanoparticles of defined composition, a cellular immune response can be induced. Aluminum adjuvants currently on market do not induce cellular immunity. Due to the well-known safety and efficacy profile and FDA approval of aluminum adjuvants for human vaccine use, the regulatory environment is favorable for this nanoparticle formulation's approval.

The presently disclosed alum adjuvant could be used as either a prophylactic or therapeutic vaccine for infectious disease, cancer, or other targets requiring cellular immunity for immunological protection. A stable nanoparticle formulation has been generated that can drain to the lymph nodes, penetrate, and retain while activating local dendritic cells. Specific aims of the presently disclosed subject matter are provided herein below.

### A. Expand the Utility of Aluminum Adjuvants

As noted hereinabove, despite clinical use of aluminum adjuvants since 1934, these adjuvants have been plagued by little Th1 induction and poor immune memory, thereby excluding these adjuvants from immunotherapy. Physical presentation of alum may play a large role in the type of elicited immune response. Clinical preparation of alum and protein by bulk mixing yields 1-20 μm polydisperse aggregates that serve as an antigen depot and induce NLRP3/NALP3 inflammasome and IL-1β signaling upon uptake.

Recent studies demonstrate that alum salts fabricated as 100-nm to 200-nm nanoparticles (NP) with surface adsorbed antigen induces cellular immunity. These approaches have numerous limitations, including but not limited to, alum's protein-specific adsorption limits due to protein pI, charge residue distribution, and molecular weight (MW), as well as alum:protein complex aggregation and protein release upon physiologically relevant ionic strengths.

Aggregation after antigen adsorption, however, limits the ability to study size-dependent effects of alum NPs. All reported alum NPs adsorb proteins on their surface, facilitating loss of tertiary structure due to electrostatic and hydrophobic interactions, limiting antigen encapsulation efficiency (EE) and loading level (LL), and limiting strength of immune response, which correlates directly to strength of antigen adsorption. Despite these findings, the exact physical presentation (e.g., size, shape, crystallinity, surface chemistry, aspect ratio) is unknown due to contradictory literature reports. A NP platform that allows for compositional control of Alum adjuvants will shed light into the mechanisms used by alum to elicit a Th1 response.

### B. Flash Nanocomplexation (FNC) Method for Banoparticle (NP) Compositional Control

Through the manipulation of formulation parameters of the FNC including volumetric flow rates, concentrations of alum salts, HA-SH MW and thiolation, degree of disulfide crosslinking, and surface modifications, it can be demonstrated that FNC provides exquisite control over NP physicochemical properties including composition, morphology, and size, while remaining uniform and scalable. FNC offers numerous unique advantages over traditional preparation methods including high pay-load LL (>20% w/w), tunable size and morphology, scalability, and flexibility given the FNC system relies on electrostatic interactions under turbulent mixing conditions to form uniform NPs.

### C. Lymph Node (LN)-Targeted NP Vaccines

It has previously been shown that smaller NPs traffic more efficiently to the local LNs following s.c. or intradermal (i.d.) injection, and this mode of delivery leads to superior DC maturation and CD4+ and CD8+ T cell activation and proliferation compared to larger NPs that cannot passively drain to the LNs. These findings suggest that to maximize LN-targeting efficiency, it is important to have a NP preparation method that has the ability to control NP size and uniformity. Currently available methods, however, suffer from poor scalability, low EE and LL, and high batch-to-batch variation. Given the difficulty associated with s.c. and i.d. administrations of vaccines, these LN-targeting NPs can likewise be administered by clinically preferred intramuscular (i.m.) route where they can likewise drain to the local muscle-draining LNs, albeit with less efficiency. Accordingly, the presently disclosed methods can likewise be applied to i.m. administration of these NPs.

### D. Redox-Triggered Alum Release

The LN microenvironment is highly reducing and its reducing activity increases after the induction of an immune response. This reducing environment provides an environmental trigger for nanotherapeutics or NP vaccines. Herein, without wishing to be bound to any one particular theory, it is thought that using HA-SH stabilized Alum:OVA NPs that can achieve small, uniform and stable NP size populations due to HA-SH stabilization of Alum:OVA complexes and disulfide bridge formation upon oxidation after aeration, for example, aeration for 12 to 72 h.

The resulting HA-SH/Alum:OVA NPs are redox sensitive. Upon reaching the LNs and crossing the subcapsular sinus macrophage border by using trimannose sugar modifications, these NPs have an abundant access to immature DCs within the LN. These NPs are either uptaken by these DCs and mediate a Th1 response by virtue of reduction of disulfide bonds within the late endosome/early lysosome reducing environment, rupture of endosome/lysosome via released alum, and subsequent antigen cross-presentation on MHC-I. At the same time, these HA-SH/Alum:OVA

NPs can be designed to trigger dumping of Alum:OVA complexes within the lymph upon exposure to the LN reducing environment, stimulating a much more robust immune response compared to Alum:OVA alone which resides at the injection site due to its high positive surface charge and large aggregate size.

### E. Antigen Availability in Key LN Compartments

The availability of antigen in the LN has been shown to be a key determinant in driving Tfh and germinal center B-cell expansion and generation of high affinity antibodies by somatic hypermutation. The duration of antigen availability in the LN as delivered and presented by NPs and the resulting immune response is not well studied, however. The LN sieves particulates by size where <70 kDa (approximately 5-nm hydrodynamic diameter) particles can pass the subcapsular sinus border lined with macrophages and travel into the LN paracortex and medulla. This subcapsular macrophage border normally excludes NPs and so NPs do not pass into the LN medulla and paracortex and pass through the LN by means of the sinuses, limiting the efficacy of NP vaccines due to lack of access of immature DCs within the LN. A recent study using NPs conjugated with tri-mannose moieties showed active transport of NPs across the subcapsular sinus border mediated by the lectin pathway of complement activation and complement receptors on the subcapsular macrophages. This insight opens up possibilities of studying antigen availability within the LN when delivered in NP form. The presently disclosed subject matter, in part, assesses how this delivery pathway along with other surface chemistry modifications changes the immune response elicited by alum NPs.

### F. Approach

NP delivery to the LNs has been shown to enhance the efficacy of subunit vaccines, yet it is unclear what NP size is optimal for alum NPs for LN targeting and maximal immunogenicity due to the wide array of alum NP sizes, shapes, and aspect ratios reported in the literature. The ideal composition of alum NPs is likewise not clear due to contradicting claims in the literature including the necessity of aluminum salt crystallinity for reactive oxygen species (ROS) production, NLRP3/NALP3 inflammasome activation, and IL-1β (3 secretion.

The presently disclosed FNC system can produce small, uniform NPs that can encapsulate proteins with a wide range of physicochemical properties with aluminum hydroxide in a scalable, reproducible manner. This system allows for subsequent modifications of the NP including coatings and NP surface modifications. This characteristic affords a high degree of control, allowing for the first thorough study of alum NP size and composition on Th1/Th2 immune response polarization. Combined with the safety record and successful history of FDA-approved alum adjuvants, this platform addresses a significant need in the immunotherapy field for Th1 adjuvants using an already approved adjuvant and so has a clear route for clinical translation.

In some embodiments, the presently disclosed subject matter aims to co-encapsulate proteins, alum salts, and HA-SH into size-controlled NPs using the FNC method; vary NP composition; and evaluate the physical properties, release profiles, immune cell stimulation, and immunogenicity mechanism of action of the HA-SH NPs.

More particularly, the presently disclosed subject matter aims to use the FNC system to thoroughly study the effect of NP composition and presentation of antigen and alum salts for the induction of Th1 and Th2 responses.

To best assess the effects of the presently disclosed NP formulations, ovalbumin (OVA) can be used as a model protein due to the availability of OVA-specific transgenic mice, immunobiological tools, and the extensive immunoengineering literature using this model to establish and characterize immunological interventions. To mimic peptide antigens, SIINFEKL (OVA 257-264) H-2Kb-restricted OVA MHC-I epitope and ISQAVHAAHAEINEAGR (OVA 323-339) I-Ad-restricted OVA MHC-II epitope for Th1 and Th2 responses, respectively, can be used. The insights gained from the whole OVA and MHC-restricted OVA peptides models will be generalizable to other protein antigen targets including whole proteins or patient-specific neoantigen peptides.

It has previously been demonstrated that the FNC platform allows for the encapsulation of peptides for application in foot and mouth disease (FMD). This FNC platform provides flexibility in the workflow for NP synthesis. The Alum:OVA complexes can be produced using a multi-inlet vortex mixer (MIVM), and then in a second step, coat the complexes with HA-SH for stabilization. Alternatively, all components can be mixed together, resulting in Alum:OVA complexes that are then coated by HA-SH in a one-pot reaction. These two means of fabricating the NP also may allow for tuning the architecture of the Alum:OVA complex within the HA-SH coating where one single Alum:OVA complex is surrounded by HA-SH or multiple complexes are coated by HA-SH.

Without wishing to be bound to any one particular theory, it is thought that FNC will encapsulate OVA and OVA MHC-restricted peptides within small 20-nm to 60-nm NPs with high EE and LL, and the resulting NP composition will skew the immune response toward Th1 by facilitating HA-SH/Alum:OVA uptake, reducing environment induced decomplexation and dumping of Alum:OVA from HA-SH, endosomal/lysosomal escape, OVA proteasomal degradation and cross presentation on MHC-I, and OVA-specific CD8+T-cell clonal expansion. Further, these small NPs will have the ability to drain to the LN, providing an enhanced immune response.

To prepare and characterize FNC-generated NPs for the co-delivery of HA-SH, Alum, and OVA, the electrostatic interactions between the alum salt, OVA protein or peptide, and HA-SH will be relied on to form a stabilized complex after turbulent mixing in a MIVM. To achieve this end, the component concentration and mass ratios, volumetric flow rate, alum salt type, pH, buffer, and ionic strength can be tuned in a single-step or two-step process. The MW of the HA-SH, the HA-SH thiolation degree (5-30%), ratio of HA to HA-SH, and electrostatic interaction strength between the HASH and Alum:OVA complex also can be tuned. The range of these parameters can be selected based on experience with previously reported FNC NP systems.

The size, size distribution and zeta potential of the NPs can be measured using dynamic light scattering (DLS) and morphology can be assessed using TEM. The EE and LL of the protein can be determined with Micro Bicinchoninic Acid (microBCA™) or NanoOrange® protein assays, and sodium dodecyl sulfate polyacrylamide gel electrophoresis (SDS-PAGE). The release of OVA from the NPs can be measured in 1×PBS (pH 7.4) medium at 37° C. containing either 15-mM dithiothreitol (DTT) or a gradient of 2.5, 25, 250 hyaluronidase (HAse) to mimic protein release after exposure to the LN reducing environment and presence of trace HAse in the skin or LN.

X-ray powder diffraction (XRD), thermogravimetric analysis (TGA), and Fourier transform infrared (FTIR) spectrometry can be utilized to assess NP crystallinity, hydroxyl content, and structure, respectively. The stability of the NPs can be monitored in water, 1×PBS (pH 7.4), 10% FBS, and 10% FBS containing supplemented Dulbecco's Modified Eagle Medium (DMEM) by measuring the size change over time by DLS. Those NP formulations with a small size that allows for rapid (hours) and slow (days) release time scales after HAse and reducing environments will be selected for subsequent activity, LN trafficking, and immunization studies.

The adjuvant activity of these NPs can be characterized using myocytes, DCs, THP-1, and HEK-Blue™ hTLR4 cells in vitro. To assess the adjuvant activity of these NPs, reporter cell lines human monocyte THP-1 NF-κB and HEK-Blue™ hTLR-4 cells (Invivogen) can be used. The THP-1 cells monitor for NF-κB signal transduction pathway using an inducible secreted embryonic alkaline phosphatase (SEAP) reporter gene that cleaves a substrate, yielding a colored product that can be quantified using a UV-VIS spectrophotometer. Similarly, the HEK293-Blue hTLR-4 cells expresses human TLR4, which activates IL-12p40 and an IL-12p40 minimal promoter fused to NF-κB binding sites, leading to production of SEAP that likewise yields a colored product upon addition of an appropriate substrate. THP-1, THP-1 with NLRP3 inflammasome knockout (THP-1defNLRP3), and HEK-Blue™ hTLR4 cells can be used to determine the adjuvant effect of each separate NP component and the synergistic effect of each NP component together or in combination. The cytokine release from myocytes, bone marrow derived CD11c+ DCs (CD11c+ BMDCs), and spleen-derived CD11c+ DCs (CD11c+ SDCs) after NP stimulation can be determined using enzyme-linked immunosorbent assay (ELISA) for TNF-α, IL-6, and IL-1β. Additionally, cytotoxicity of NPs and NP components can be assessed in all cell lines using alamarBlue® assay.

Further, the effect of NP composition on DC uptake, maturation, and subsequent stimulation and expansion of OT-1 and OT-II OVA-specific T-cells will be characterized. More particularly, the effect of NP composition on bone marrow derived CD11c+ DCs (BMDCs) and spleen derived CD11c+ DCs, and monocyte derived macrophages due to inherent variability and poor in vitro to in vivo immunological correlation found in established DC or macrophage cell lines will be characterized. DC uptake and presentation can be determined by labeling the protein with NIR Cy5.5 dye and observing co-localization in endosomes (EEA1), lysosomes (LAMP1), or cytosol using confocal microscopy. DC activation and antigenic presentation can be determined by flow cytometry measurement of CD40, CD80, CD86, MHC-I, and MHC-II. The stimulatory capacity of treated DCs can be assessed by DC co-culture with OVA-specific OT-I CD8+ or OT-II CD4+ primary T-cells stained with carboxyfluorescein diacetate succinimidyl ester (CFSE), and T-cell generations counted by flow cytometry. NP formulations that induce the greatest OT-I CD8+ T-cell proliferation will be chosen for further study.

Based on the results from these studies, the relationship between FNC formulation parameters and NP properties, as well as NP composition with DC maturation and subsequent T-cell activation can be mapped. Without wishing to be bound to any one particular theory, it is thought that packaging alum with HA-SH will increase the immunogenicity of Alum and serve to skew the immune response toward a cellular immune response. Further, controlling the presentation and form of alum within NPs instead of microparticle sized aggregates used in current vaccinations, the immune response can be skewed by virtue of differing physical presentation.

If the size of HA-SH/Alum:OVA NPs cannot be tuned due to the need for thiolated HA to form stable, crosslinked NPs, HA of the same molecular weight can be doped in the NPs and the relative amounts of HA:HA-SH can be varied to tune the NP size while keeping the NP stable. If the disulfide crosslinked NPs are too stable in reducing conditions or with incubation with HAse, HA can be added in with HA-SH to achieve a formulation that is responsive to these conditions within an appropriate timeframe. Although problems with the phenotypic characterization of DC differentiation and maturation by flow cytometry are not anticipated, an alternative would be with ELISA cytokine analysis of the supernatant of DCs isolated from LNs.

In other embodiments, the presently disclosed subject matter aims to modify FNC NPs with targeting ligands to control NP targeting to specific LN compartments and cell types, and to evaluate the immune response of these NPs in healthy and tumor bearing male and female mice.

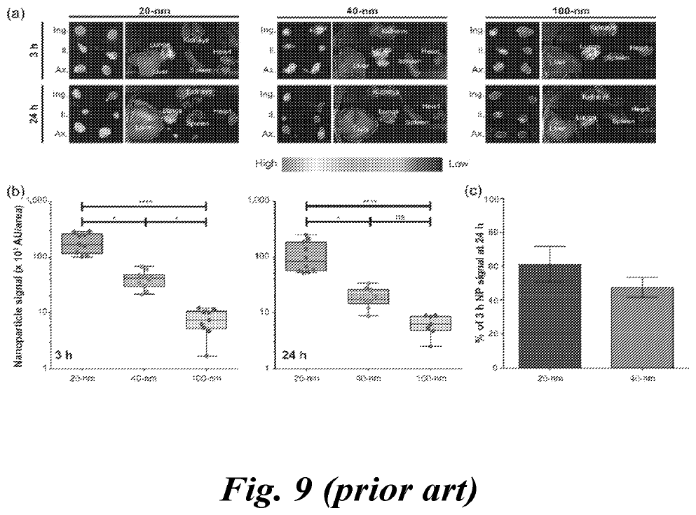

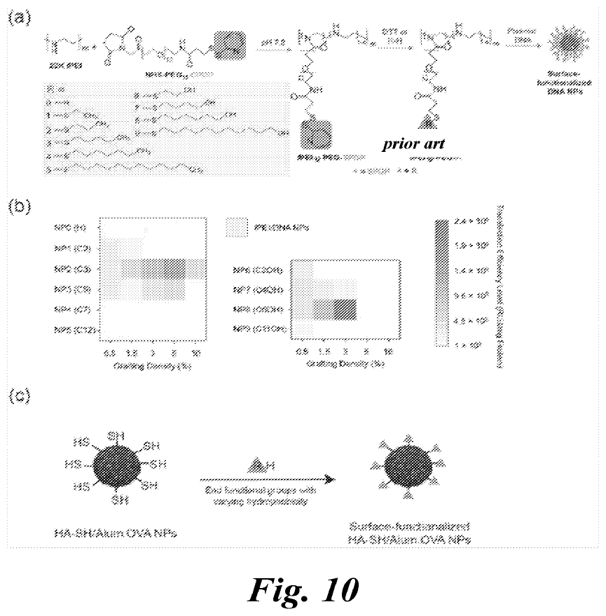

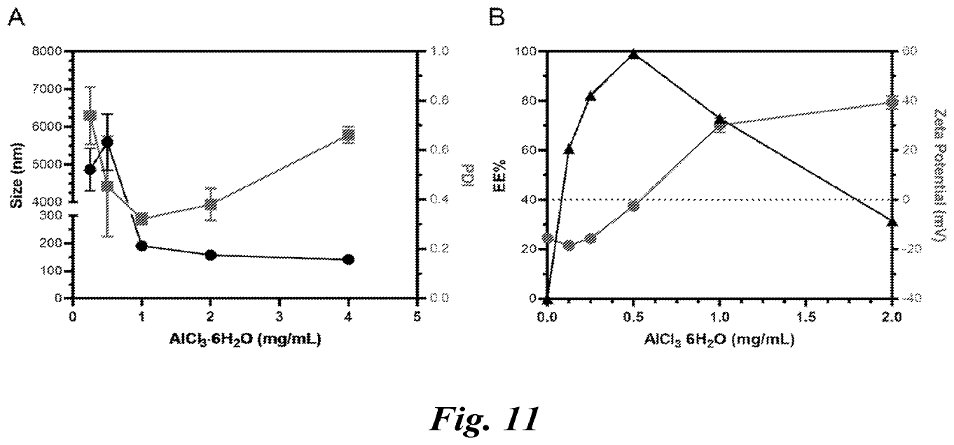

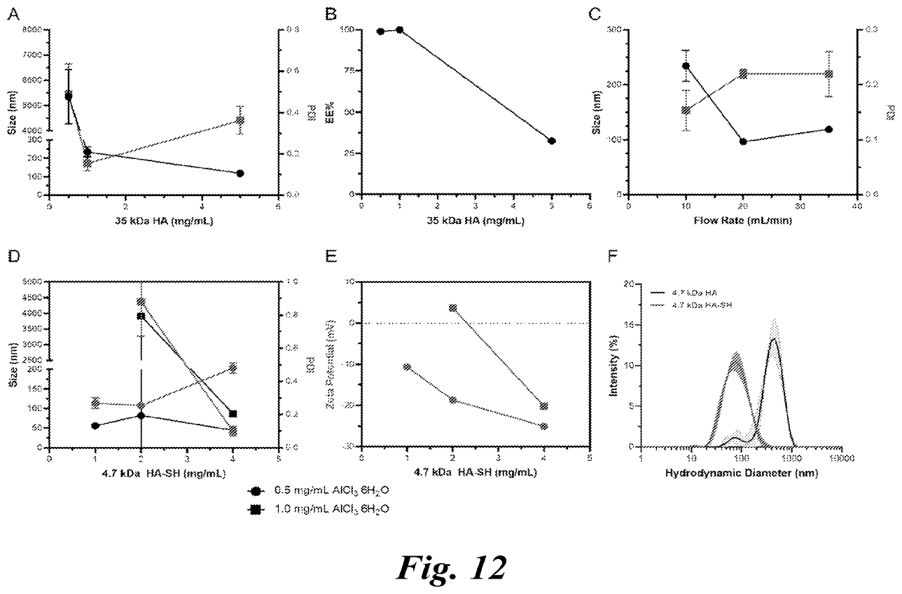

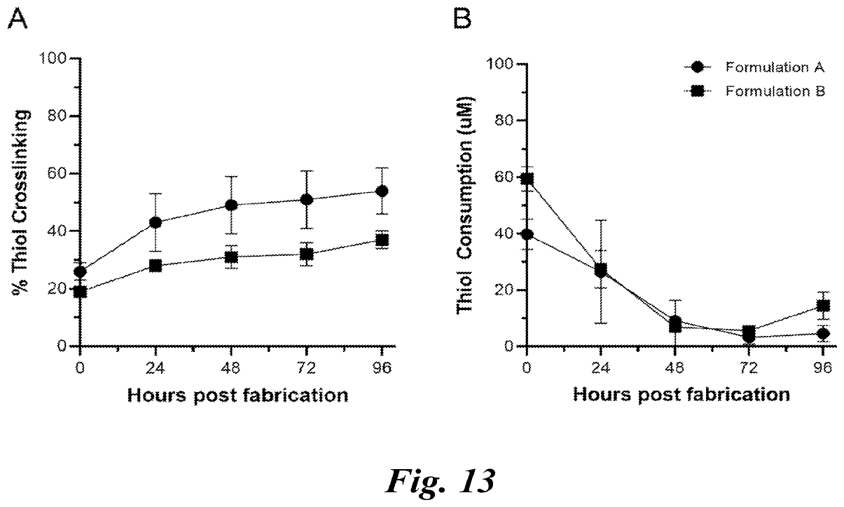

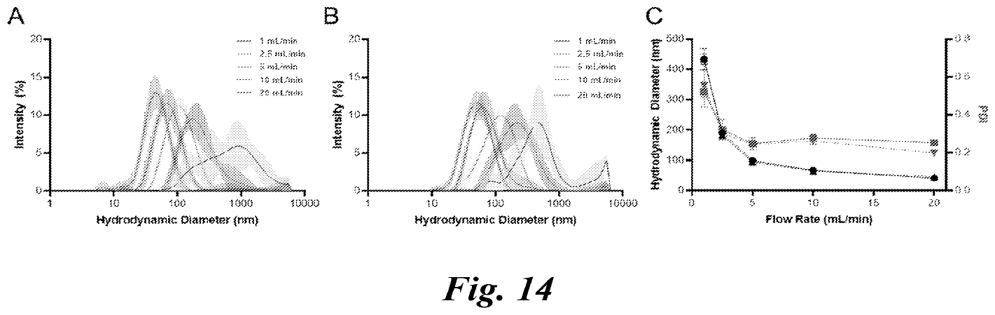

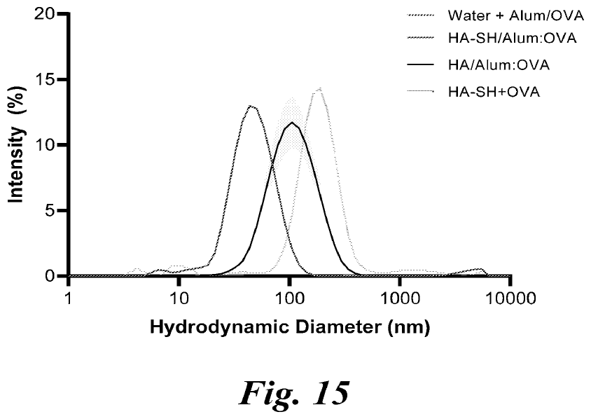

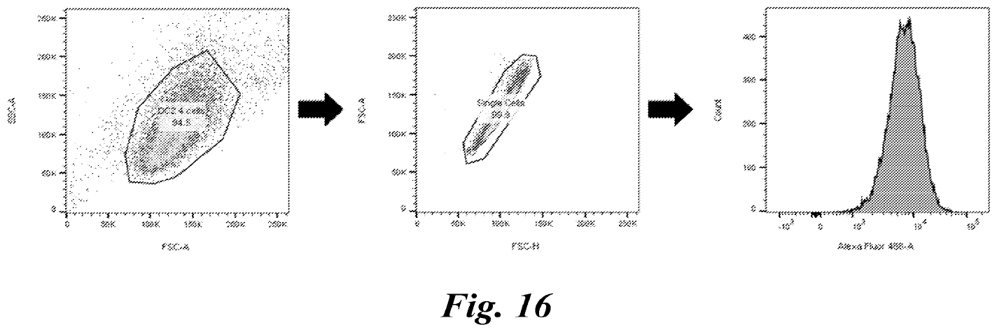

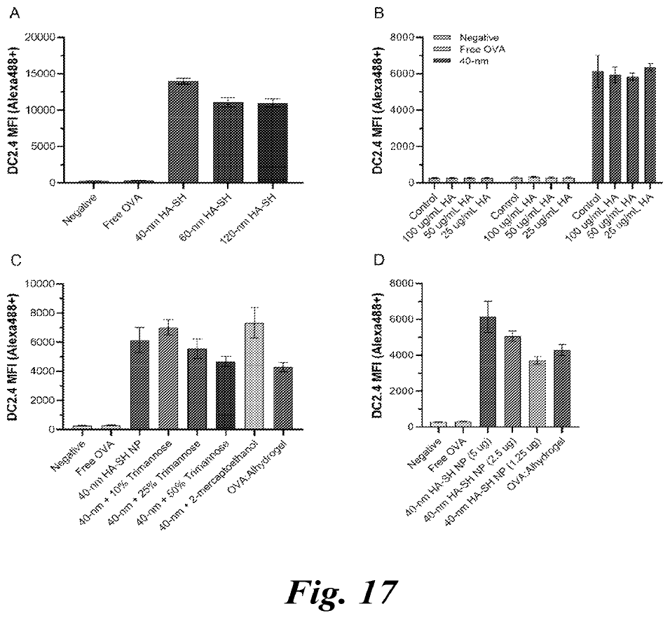

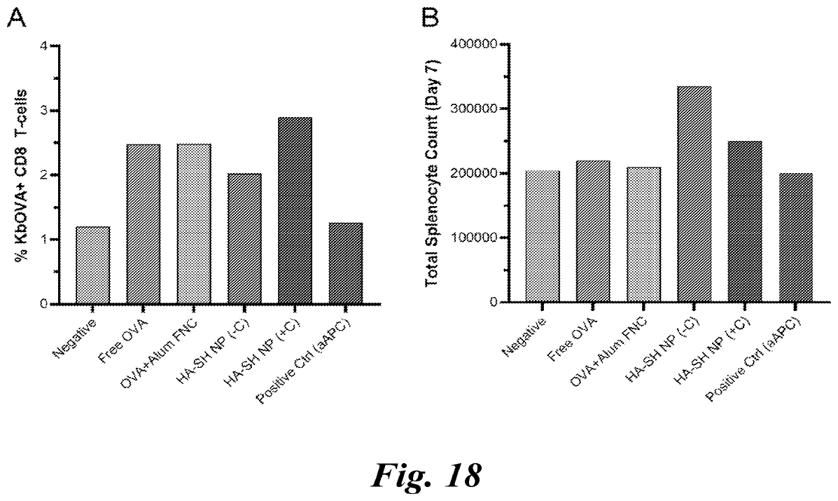

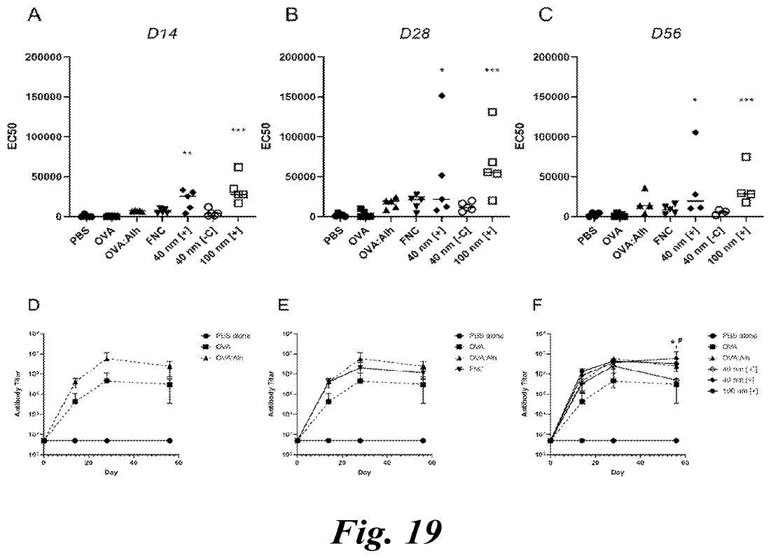

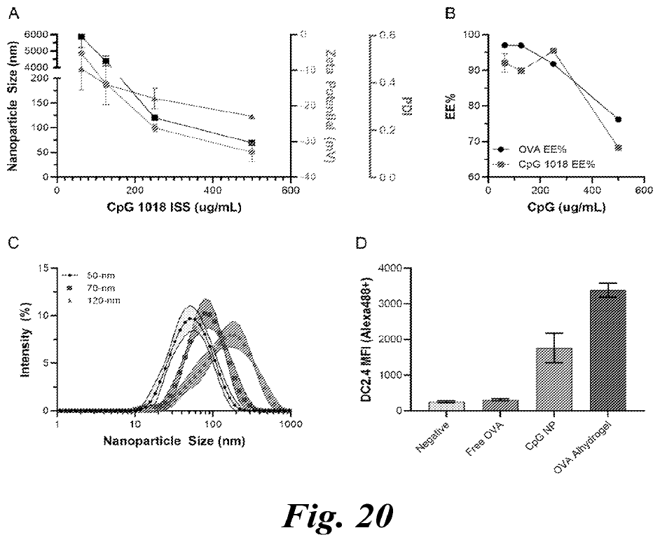

It has been previously shown that NP delivery and LN targeting enhance the efficacy of subunit vaccines. The presently disclosed FNC system will encapsulate OVA, alum, and HA-SH within one NP in a diverse array of possible architectures, morphologies, surface charge, and surface chemistry. Optimizing this system will allow for selective skewing of the immune response to Th1 or Th2 using the same alum adjuvant but only changing the physical presentation of this adjuvant, thereby expanding alum's utility and demonstrating the importance of physical presentation architecture in eliciting an immune response. These NPs will co-deliver OVA with alum to the draining LN and subsequently to a high density of DCs and other antigen-presenting cells (APCs) (FIG. 9). By tuning the formulation, release kinetics and antigen availability in the LN can be controlled, providing further control of the immune response. Further tuning the surface chemistry of these NPs will allow for finer skewing of the immune response. It has been shown that tuning surface chemistry and hydrophobicity of NPs augments their transfection efficacy, and it is anticipated a similar finding for the immune response (FIG. 10).

Without wishing to be bound to any one particular theory, it is thought that LN co-delivery of OVA and alum with controlled physical presentation and chemistries will activate DCs, other innate immune cells (e.g., neutrophils, macrophages, monocytes), and lead to antigen-specific T cell immune response in the draining LN. Finally, the utility of this platform using in vivo adoptive cell killing assays and orthotopic melanoma B16-OVA model also will be demonstrated.

To this end, the LN-targeting effect and biodistribution of the NPs can be assessed in 6-8 week-old C57BL/6 male and female mice. After s.c. injection at the tail base compared to i.m. at the right hind calf, whole-body imaging can be taken at 0.5, 2, 6, 12 and 24 h post-injection, and then daily thereafter until the signal is undetectable. At the time of highest fluorescence intensity, the main organs including heart, liver, lung, spleen, kidney, i.m. or s.c. injection site, and major LNs (inguinal, iliac, axial) can be harvested for ex vivo imaging.

It has previously been shown that poly(lactic-coglycolic acid)-block-poly(ethylene glycol) (PLGA-b-PEG) NP size tightly regulates their drainage to LN, with the population of 20-60 nm NPs mostly responsible for quick LN drainage within 2 h following s.c. injection (FIG. 9). It is not clear if similar size bias for LN drainage will be present in this alum NP system due to differences in NP material, stiffness, and surface charge. HA-SH with Cy5.5 and OVA can be labeled with Cy7.5 to determine NP in vivo stability and NP LN trafficking kinetics by in vivo imaging and qualitative LN compartment localization by confocal microscopy after LN cryosectioning and immunofluorescence staining (e.g. CD3+T-cells, B220+ Bcell, CD123+ plasmacytoid DCs (pDCs), CD207+ Langerhans DCs, CD8α+ DCs, CD11b+ DCs, CD11c+ DCs, and F4/80+ macrophages). Of particular interest is CD8α+ DCs, which are the primary DC population that cross-presents antigen to CD8+ T-cells to initiate a Th1 response, while CD11b+ DCs are one of the main subsets involved in MHC-II presentation to CD4+T-cells. Mice treated i.m. with free OVA mixed with Alhydrogel® (Invivogen) to form 1-20 μm aggregates and no treatment will be controls. Five animals per group can be used to detect 40-50% variance with a statistical power of 80% and 5% Type I Error (α).

Other embodiments will be directed toward modifying NPs with surface functional groups and targeting ligands for targeting immune cell populations and lymph node compartments, and to study the effects of these modifications on the immune response in healthy mice. To target macrophage, DCs, specific LN compartments, and germinal centers (GCs), mannose sugars (e.g., tri-mannose [4-Aminophenyl 1,3-α-1,6-α-D-mannotrioside]) will be conjugated onto the NP surface. These NPs will be further modified by surface conjugation of a library of functional groups (e.g., primary amine, hydroxyl, carboxylic acid, sulfate) with varying alkyl chain lengths to modulate hydrophobicity for modification of the immune response. The LN trafficking (see hereinabove) and immune response of these modified and pristine NP formulations will be assessed in 6-8 week old male and female C57BL/6 mice. To assess the immune response in healthy mice using these NPs, mice can be injected with NP formulations by i.m. or s.c. administration that skew toward Th1 or Th2 response on day 0. Blood can be collected at days −1, 7, 14, 21, and 28 for antibody titer, isotype (IgM, IgG, IgA) and subtyping analysis to measure Th1/Th2 polarization (IgG1/IgG2a). From a subset of mice, spleens can be collected at days 7 and 30 for ELISpot measurements of T-cell polyfunctionality (IFNγ, TNF-α, IL-2), Th1 response (IFNγ, IL-2), and Th2 response (IL-4, IL-5). At day 28, LNs can be collected, cryosectioned, and immunofluorescently stained (see hereinabove for full staining panel) for GL-7 to assess GC formation and CXCR5/PD-1 for CD4+ Tfh expansion. It has been demonstrated that after alum administration in rhesus macaques, neutrophils and monocytes are recruited to the site of injection, uptake antigen, and present antigen primarily on MHC-II to induce CD4+ T-cell response. This mechanism of antigenic presentation can be gauged by collecting the s.c. and i.m. injection sites and LNs at days 1, 7, and 14 to assess the recruitment of and subsequent LN trafficking of innate immune cells Ly6G+ neutrophils, CD11bhi CD115hi monocytes, CD123+ pDCs, CD207+ Langerhans DCs, CD8a+ DCs, CD11b+ DCs, and CD11c+ DCs by immunofluorescence or immunostaining with hematoxylin and eosin (HE) staining. These immune subsets can be isolated and their presentation of OVA gauged by flow cytometry for MHC-I (SIINFEKL) and MHC-II (ISQAVHAAHAEINEAGR). For the NPs with optimal surface chemistries that provide a Th1 response, the ability of these NPs to induce cell-killing can be assessed in a SIINFEKL-splenocyte adoptive transfer model. C57BL/6 mice can be vaccinated s.c. 1 cm from the tail or i.m. at right calf on day 0 and day 7. Vaccinated and naïve mice can be injected with a 1:1 mixture of splenocytes, half of which are incubated with SIINFEKL peptide and stained with a high level of CFSE and the other with tenfold lower level of CSFE. Spleens can be harvested 18 h after transfer and transferred splenocytes analyzed by flow cytometry to assess degree of cell killing. Three NP formulations that give the strongest Th1 polarized response will be selected for subsequent immunological testing. For each of the above animal studies, 5-10 mice will be used to obtain a statistical power of 80% with a Type I Error (α) of 5%.

To determine NP effect on survival of B16-OVA tumor-bearing mice in prophylactic and therapeutic vaccination models, the anti-tumor activity of the three strongest Th1-inducing NPs using a prophylactic and therapeutic orthotopic melanoma B16-OVA model can be determined. For the prophylactic model, C57BL/6 mice can be administered with NP vaccine s.c. tail-base or i.m. right flank on days −14 and −7. On day 0, mice can be injected with 2×106 B16-OVA cells s.c. left flank. Tumor volume can be monitored until day 30. For the therapeutic model, C57BL/6 mice can be administered with 2×10⁶ B16-OVA cells s.c. left flank at day 0. On days 7 and 14 the respective NP vaccines can be administered and tumor volume monitored until day 30.

To this end, the effect of NP composition on inducing Th1 versus Th2 response by targeted delivery of the HA-SH/Alum:OVA NPs to the LN, bypassing the subcapsular macrophage lining into the paracortex and medulla regions by tri-mannose modification, targeted uptake by macrophages and dendritic cells using sugar modifications, and effects of surface functional groups and alkane chain length (hydrophobicity) on NP immunogenicity can be demonstrated.

All animal models and methods for assessment of NP biodistribution and immune response have been developed and demonstrated in previous studies. HA-SH/Alum:OVA NPs within the reducing environment of the LN will likely become reduced, leading to dumping of the Alum:OVA complexes within the LN. If the LN targeting efficiency is high and most of the dose makes it to the LN, then an excess of Alum:OVA complex may lead to strong inflammation and, if the persistence is long and antigen availability is high, anergy or deletion of OVA-specific T-cells and B-cells. Anergy can be assessed by isolating T-cells from LNs and spleen at days 14, 28 for flow cytometry analysis of CD4+ and CD8+T-cell exhaustion by measuring PD-1, LAG-3, TIM-3, 2B4, CD160. This measurement may be especially problematic after inclusion of tri-mannose sugars, which give NPs access to the paracortex and medulla space. To counter this possibility, the thiolation degree of the HA-SH can be lowered or HA can be added in to lower the responsiveness to redox environments. If the disulfide bond of the functional groups is found to release these surface chemical modifications prior to drainage to the LN and subsequent immune response initiation, maleimide-thiol chemistry can be adopted for functional group conjugation.

For all vaccination studies, C57BL/6 mice, the standard model for Th1 cellular immune response for assessing induction and expansion of antigen-specific cytotoxic T-cells, will be used. All mice will be 6-8 weeks old as is standard in the literature for assessing immune response. Male and female mice also will be used to determine sex differences in induction of an OVA-specific immune response initiated by HASH/Alum:OVA NPs and controls. All mice will receive 10 μg OVA dose. To assess statistical significance, studies such that at least 80% power is achieved with Type I Error (α) of 5% can be designed. Given the high variability in biodistribution and immunological studies, it is typical for these studies to have 5-10 mice per group. Pilot studies will be performed to confirm that an appropriate number of mice are included for the resulting effect size and to achieve >80% power. ELISA log₁₀ OVA-specific antibody titers, ELISpot T-cell cytokine secretion, flow cytometry assessments, and survival will be compared using ANOVA followed by Tukey's post-hoc test or Krusal-Wallis test with Dunn's Multiple Comparison post-hoc test between groups depending upon the data's normality.

### G. Representative Embodiments

In some embodiments, the presently disclosed subject matter provides a nanoparticle comprising an alum core and a coating, wherein the nanoparticle has a number average size between about 20 nm and about 300 nm and a polydispersity index between about 0.1 to about 0.3.

In some embodiments, the nanoparticle has a number average size of about 20 nm, 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, and 300 nm. In some embodiments, the nanoparticle has a number average size of less than 100 nm, including 20 nm, 30 nm, 40 nm, 50 nm, 60 nm, 70 nm, 80 nm, 90 nm, 100 nm. In certain embodiments, the nanoparticle has a number average size between about 20 nm and about 60 nm. In particular embodiments, the nanoparticle has a number average size between about 40 nm and about 50 nm.

In particular embodiments, the alum core comprises an aluminum compound selected from the group consisting of aluminum hydroxide, aluminum phosphate, aluminum chloride, amorphous aluminum hydroxyphosphatesulfate (AAHS), potassium aluminum sulfate, and combinations thereof.

In some embodiments, the surface coating is selected from the group consisting of one or more anionic polysaccharides, one or more cationic polymers, and one or more anionic polymers.

In some embodiments, the one or more anionic polysaccharides is selected from the group consisting of hyaluronic acid, heparin sulfate, chondroitin sulfate, and dextran sulfate.

In some embodiments, the one or more cationic polymers are selected from the group consisting of linear or branched polyethylenimine, poly(L-lysine), poly(β-amino esters), protamine, chitosan, and combinations thereof.

In some embodiments, the one or more anionic polymers comprise cytosine phosphoguanosine (CpG) oligodeoxynucleotide.

In certain embodiments, the surface coating is crosslinked. In some embodiments, the crosslinking is a reversible crosslinking, such as crosslinking with disulphide bridges. One of ordinary skill in the art would recognize that other reversible or irreversible crosslinking chemistries known in the art are suitable for use with the presently disclosed subject matter including, but not limited to, maleimide/thiol, acrylate, click chemistry, and others on the hyaluronic acid or polysaccharide backbone.

In more particular embodiments, the crosslinked surface coating comprises thiolated hyaluronic acid. In other embodiments, the crosslinked surface coating comprises an unmodified hyaluronic acid (i.e., a low molecular weight hyaluronic acid, e.g., <300 kDa, or a high molecular weight hyaluronic acid above 300 kDa).

In certain embodiments, the nanoparticle further comprises a protein or peptide antigen entrapped within the anionic polysaccharide coating.

In certain embodiments, the nanoparticle further comprises a protein or peptide antigen conjugated to a surface of the anionic polysaccharide coating.

In certain embodiments, the nanoparticle has a size between about 20 nm and about 200 nm.

In other embodiments, the presently disclosed subject matter provides a vaccine adjuvant comprising a presently disclosed nanoparticle.

In other embodiments, the presently disclosed subject matter provides a vaccine comprising the presently disclosed nanoparticle or a presently disclosed vaccine adjuvant.

In particular embodiments, the vaccine comprises a vaccine selected from the group consisting of Anthrax, DT, DTaP (Daptacel), DTaP (Infanrix), DTaP-IPV (Kinrix), DTaP-IPV (Quadracel), DTaP-HepB-IPV (Pediarix), DTaP-IPV/Hib (Pentacel), HepA (Havrix), HepA (Vaqta), HepB (Engerix-B), HepB (Recombivax), HepA/HepB (Twinrix), HIB (PedvaxHIB), HPV (Gardasil9), Japanese encephalitis (Ixiaro), MenB (Bexsero,Trumenba), Pneumococcal (Prevnar13), Td (Tenivac), Td (MassBiologics), Tdap (Adacel), Tdap (Boostrix), and malaria (RTS.S (Mosquirix))

In some embodiments, the vaccine is a cancer vaccine. In particular embodiments, the cancer vaccine is selected from the group consisting of BiovaxID (follicular lymphoma, a type of non-Hodgkin's lymphoma), sipuleucel-T (prostate cancer), oncophage (kidney cancer), and talimogene laherparepvec (melanoma), or a patient-derived neoantigen.

In other embodiments, the presently disclosed subject matter provides a method for treating a disease or condition, the method comprising administering a presently disclosed nanoparticle to a subject in need of treatment thereof.

In some embodiments, the treating is prophylactic. In other embodiments, the treating is therapeutic.

In certain embodiments, the nanoparticle drains to one or more lymph nodes.

In some embodiments, the nanoparticle induces a Th1 anti-tumor response.

In some embodiments, the administering of nanoparticle is selected from the group consisting of intradermal (i.d.), subcutaneous (s.c.), and intramuscular (i.m.).

In some embodiments, the presently disclosed subject matter provides a medicament comprising a presently disclosed nanoparticle. In some embodiments, the medicament further comprises a vaccine and/or a vaccine adjuvant comprising a presently disclosed nanoparticle.

In some embodiments, the presently disclosed subject matter provides for the use of a vaccine comprising a presently disclosed nanoparticle or a vaccine adjuvant comprising a presently disclosed nanoparticle for treating or preventing an infectious disease, a cancer, and/or one or more other targets requiring cellular immunity for immunological protection. In certain embodiments, the use is prophylactic or therapeutic.

In other embodiments, the presently disclosed subject matter provides a method for preparing an alum nanoparticle, the method comprising admixing alum with a protein or peptide antigen in a flash nanocomplexation apparatus. In certain embodiments, the method further comprises admixing a surface coating with the alum and protein or peptide antigen in a flash nanocomplexation apparatus. In certain embodiments, the method further comprises a one-step flash nanocomplexation process or a two-step flash nanocomplexation process.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly an alum NP and one or more additional therapeutic agents. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the alum NPs described herein can be administered alone or in combination with further adjuvants that enhance stability of the NPs, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a presently disclosed alum NP and at least one additional therapeutic agent can be varied so long as the beneficial effects of the combination of these agents are achieved. Accordingly, the phrase “in combination with” refers to the administration of a presently disclosed alum NP and at least one additional therapeutic agent either simultaneously, sequentially, or a combination thereof. Therefore, a subject administered a combination of a presently disclosed alum NP and at least one additional therapeutic agent can receive an alum NP and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the alum NP and at least one additional therapeutic agent are administered simultaneously, they can be administered to the subject as separate pharmaceutical compositions, each comprising either an alum NP or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound of formula (I) and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

Qₐ/Q_{A}+Q_{b}/Q_{B}=Synergy Index (SI)

wherein:

Q_{A} is the concentration of a component A, acting alone, which produced an end point in relation to component A;

Qₐ is the concentration of component A, in a mixture, which produced an end point;

Q_{B} is the concentration of a component B, acting alone, which produced an end point in relation to component B; and

Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

As used herein, the term “inhibit,” and grammatical derivations thereof, refers to the ability of a presently disclosed alum NP, to block, partially block, interfere, decrease, or reduce the occurrence or symptom of a disease or condition. Thus, one of ordinary skill in the art would appreciate that the term “inhibit” encompasses a complete and/or partial decrease in occurrence of symptom of a disease or condition, e.g., a decrease by at least 10%, in some embodiments, a decrease by at least 20%, 30%, 50%, 75%, 95%, 98%, and up to and including 100%.

### H. Pharmaceutical Compositions and Administration

In another aspect, the present disclosure provides a pharmaceutical composition including the presently disclosed alum NPs alone or in combination with one or more additional therapeutic agents in admixture with a pharmaceutically acceptable excipient.

In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra -sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Reprogramming Alum Adjuvant to Tailor Immune Response

### 1.1 Overview

Vaccination is responsible for the eradication of smallpox and the rapid declining of many infectious diseases. To maximize safety and minimize reactogenicity, vaccine development is gradually shifting from whole, inactivated vaccines to well-defined subunit vaccines. This approach, however, often suffers from reduced immunogenicity. Development of adjuvants that are safe and potentiate immune response and long-term immune memory is paramount.

Aluminum salts (alum) are the most widely used adjuvants, yet they lack utility for vaccine targets requiring cellular immunity as the mechanistic correlate of protection due to poor stimulation of CD8 T-cells and requirement of multiple boost doses for immune memory. Recent studies demonstrate that alum salts fabricated as 100-nm to 200-nm nanoparticles (NPs) with surface adsorbed antigen induces cellular immunity. Jiang et al., 2018; Wang et al., 2015.

Aggregation after antigen adsorption, however, limits the feasibility to study size dependent effects of these alum NPs. Without wishing to be bound to any one particular theory, it was thought that alum NPs with well-defined and smaller size could yield a higher efficiency of delivery to a rich population of immature dendritic cells (DCs) within the draining lymph nodes (LNs), Wilson et al., 2003, via intradermal (i.d.d.) or subcutaneous s.c.c.). Such a platform for producing uniform and small alum NPs that allows for skewing immune stimulation toward a Th1 response, however, is not available. In this example, alum NPs with small (<100 nm) and controlled size were generated. Their effectiveness to traffic to the draining LNs was evaluated. Further, their potency as an adjuvant to elicit strong Th1 immune response via antigen cross presentation was assessed.

### 1.2 Methods

A flash nanocomplexation (FNC) process to prepare alum NPs and ovalbumin (OVA) co-loaded NPs using thiolated hyaluronic acid (HA-SH) through both electrostatic interactions and disulfide crosslinking to stabilize NPs was adopted. A set of alum NPs were generated by varying formulation parameters, and characterized by particle size, alum crystallinity, surface charge, morphology, stability, reduction sensitivity and release profile. NPs with small size (20 nm to 60 nm) were selected and DC stimulation, differentiation and maturation, and antigen-specific effector T-cell response was evaluated. These properties were correlated with Th1 response mechanism.

### 1.3 Representative Results

The tailored FNC process allowed for production of HA-SH coated alum:OVA NPs with high uniformity (PDI <0.2), small size (30 nm to 100 nm), low batch-to-batch variability, and tunable formulation. Addition of HA-SH coatings to alum:OVA complexes and subsequent crosslinking post aeration stabilized the NPs in buffered medium. In reducing conditions, these NPs underwent triggered aggregation and protein release. Due to the LNs' reducing environment post immunization, this mechanism of triggered aggregation allows for enhanced retention at the LNs post administration. Whole body imaging demonstrated the NPs' ability to drain and retain in the LN over a 48-h time period similar to previously published work, Howard, G. P. et al., 2019, and NP colocalization with key LN dendritic cell subpopulations. Furthermore, in vitro tests with bone marrow derived dendritic cells (BMDCs) and

DC2.4 DCs demonstrated a significantly higher level of maturation, as measured by CD86, CD40, and CD80 staining, and MHC I/II presentation after treatment with HA SH/OVA NPs compared to free protein, benchmark control Alhydrogel adjuvant, and FNC produced alum: OVA NPs. After screening of additional NP parameters including alum crystallinity and hydrophobicity, selected NPs with the strongest Th1 response will be used to determine the prophylactic and therapeutic anti-tumor effect in B16 OVA bearing mice.

### Example 2

### Three-Inlet FNC for Production of HA/HA-SH Coated Alum:OVA Complex Nanoparticles

Nanoparticles were generated using a three-inlet flash nanocomplexation confined-impinging jet (CIJ) device. The first inlet contained the polyanionic coating agent of varying concentration, this being the 4.7 kDa or 35 kDa hyaluronic acid (with or without 20% thiolation degree) or CpG 1018 ISS (sequence: 5′-TGACTGTGAACGTTCGAGATGA-3′ (SEQ ID NO: 1) with phosphorothioate backbone synthesized by TriLink Biotechnologies) dissolved in distilled, deionized water (conductivity <100 μS/cm and TOC <50 ppb). ISS 1018 is a short (22-mer), synthetic, unmethylated CpG oligodeoxynucleotide with immunostimulatory activity.

The second inlet contained model protein Ovalbumin (OVA) at a concentration of 250 μg/mL in 25 mM HEPES, pH 11.00. The third inlet contained varying concentrations of aluminum chloride hexahydrate (AlCl₃.6H₂O) dissolved in deionized water with pH 2.00 generated by adding 13 M HCl. The solutions were then mixed rapidly under turbulent mixing conditions using a NE-4000 Programmable 2 Channel Syringe Pump (SyringePump.com). The relative flow rates can be modulated to yield nanoparticles of controlled size (40 nm-200 nm) depending upon the nanoparticle composition and coating material. Nanoparticles are then characterized using dynamic light scattering (DLS) to measure the hydrodynamic size (intensity- or number-average), polydispersity, and zeta potential. Nanoparticles are then further processed (e.g. sterile filtered, diluted in cell media or isotonic solutions for injection, crosslinked overnight by shaking and aeration) and used for subsequent experimentation.

### Example 3

### Protocols

### 3.1 Screening Alum Complex Conditions for Small Alum: OVA Complexes by Two-Inlet FNC

To generate Protein and Aluminum Hydroxide complexes of small size, a two-inlet flash nanocomplexation (FNC) setup was used. In one inlet, model protein Ovalbumin (OVA) at 250 μg/mL was dissolved in 25 mM HEPES pH 11.00 and in the other inlet with aluminum chloride hexahydrate (AlCl₃.6H₂O) of varying concentrations (250 μg/mL-4 mg/mL) was dissolved in pH 2.00 deionized water. By fixing the flow rate at 10-mL/min and increasing the AlCl₃.6H₂O salt concentration, complexes of small size could be generated with varying zeta potentials that correspond to the AlCl₃.6H₂O salt content. The aluminum hydroxide forms during the mixing process and immediately complexes with protein. For subsequent studies involving coating of these complexes, two formulations with a fixed concentration of 250 μg/mL OVA with either 0.5 mg/mL AlCl₃.6H₂O (slightly neutral complex) or 1.0 mg/mL AlCl₃*6H₂O (strongly positive complex) were chosen for screening coatings of anionic polymers (e.g. CpG 1018 ISS oligodeoxynucleotides), carbohydrates (e.g. Hyaluronic acid), or combination thereof. Similarly, complexes composed of low AlCl₃.6H₂O concentrations can be utilized for coating of cationic polymers (e.g., linear polyethylenimine and chitosan). To determine the encapsulation efficiency of the formed complex, the Alum:OVA complexes were filtered using a 100-kDa MWCO Amicon filter, centrifuging at 8000×rpm for 90 seconds, and measured the free protein content in the filtrate by micro BCA protein assay.

### 3.2 Crosslinking and Surface Modification of HA-SH/Alum: OVA Complex Nanoparticles

Nanoparticles were fabricated on a three inlet FNC device and then aliquoted into 20-mL scintillation vials. The scintillation vial caps were left off to allow for atmospheric oxygen to crosslink the nanoparticles with shaking at room temperature. Degree of crosslinking was measured using a modified Ellman's reagent assay (Riener et al., Analytical and Bioanalytical Chemistry, 2002) and measuring the absorbance on an Infinite M200 Tecan plate reader.

To modify the nanoparticle surface, thiolated compounds (e.g., thiolated modified 4-Aminophenyl 1,3-α-1,6-α-D-mannotrioside, 2-mercaptoethanol) dissolved in DMSO are added directly to the HA-SH/Alum:OVA nanoparticle immediately after production on the FNC device and allowed to shake overnight at room temperature for crosslinking. Total volume fraction of DMSO does not exceed 0.2% v/v during conjugation step.

### 3.3 In Vitro Nanoparticle Uptake and Processing

DC2.4 cells were cultured in RPMI-1640 supplemented with 10% FBS, 2 mM L-glutamine, 10 mM HEPES, 50 μM 2-mercaptoethanol, 1X Non-Essential Amino Acids, and 1% Penicillin/Streptomycin as instructed by supplier (Millipore-Sigma). For uptake assays, cells were seeded at a density of 100,000 cells/well in 24-well tissue culture treated plate. Nanoparticles were diluted to give 2.5, 5, or 10 μg/mL OVA-FITC dosage in cell medium and 0.5 mL of the nanoparticle suspension was added to cells. At 3 h, cells were washed three times with PBS to wash away free NPs, lifted using 0.25% Trypsin-EDTA, and subsequently washed three times with PBS. The cells were then fixed using 4% paraformaldehyde for 10 minutes, washed three times with PBS, and then stored for analysis. Cells were analyzed by a BD FACSCanto and OVA uptake determined by FITC signal. Flow cytometry data was analyzed using FlowJo v 10.7.

### 3.4 Vaccination of C57BL6 Mice and Determination of Antibody Titer

Mice were immunized subcutaneously at 1 cm from tail-base twice at 14 days apart. Blood from immunized animals were collected by submandibular bleed at Days 0, 14, 28, and 56. Sera were harvested for use in total IgG ELISA assays. A 96-well plate was coated with 5 μg/mL of OVA in carbonate/bicarbonate buffer (pH 9.6) overnight at 4 ° C., and washed with 0.05% Tween 20 in 1×PBS, and then blocked with 5% skim milk in 1×PBS by incubation for 1 h at 37° C. The plate was washed again with 0.05% Tween 20/PBS and kept at 4° C. in 1×PBS before use. Sample sera from different time points were serially half-log diluted (from 500× to 1.58×10⁸×) and added to the wells of the pre-coated plate, and then incubated at 4° C. overnight. The plate was washed with 0.05% Tween 20 in 1×PBS, and horseradish peroxidase (HRP)-conjugated anti-mouse total IgG antibody (1:2000 dilution, goat anti-mouse IgG (H+L), Abcam) was added and incubated for 2 h at 37° C. The plate was given a final wash and antibody detection was developed using TMB substrate solution (Thermo Fisher) before being stopped with 1 N HCl. Absorbance was read at an optical density (OD) of 450 nm on an Infinite M200 Tecan plate reader. Endpoint titer was determined using a statistical method (Frey et al. Journal of Immunological Methods 1998)

### 3.5 In Vitro C57BL6 CD8+T-Cell Stimulation.

To isolate the splenocytes, the spleen and lymph node were harvested from a C57BL6 wildtype mouse and macerated through a cell strainer, while washed with PBS. Cells were counted with a hemocytometer and resuspended at 600,000 cells/mL in complete RPMI-1640 medium supplemented with 10% fetal bovine serum and T cell growth factor, a cytokine cocktail derived from condition media produced from stimulated human PBMC as previously described (Durai et al. Cancer Immunology, Immunotherapy, 2009).

Post harvesting and counting on day 7, cells were stained with a solution of APC-conjugated rat anti-mouse CD8a antibody, clone 53-6.7 (BD Pharmingen) at a 1:100 ratio and 10 μg of Kb-Ig loaded either with ovalbumin-specific peptide SIINFEKL or a non-cognate peptide in 100 μL of FACS wash buffer for 1 hour at 4° C. MHC Kb-Ig dimer was produced in-house as previously described (Schneck, Immunological Investigations, 2000). Cells were then washed with PBS, and then resuspended in a solution of 1:1000 of LIVE/DEAD® Fixable Green Dead Cell Stain (ThermoFisher), and a 1:350 solution of PE-conjugated streptavidin (BD Pharmingen), in 100 μL of PBS for 15 minutes at 4° C. Cells were then washed with FACS wash buffer and read on a BD FACSCalibur. Percentage of ovalbumin-specific CD8+ T cells was calculated by subtracting the percent positive for the non-cognate Kb-Ig from the cognate Kb-Ig (SIINFEKL).

### Example 4

### Prophetic Examples

4.1 Coating of Alum Complexes using Polycationic Polymers Linear Polyethylenimine and Chitosan

Using alum complexes with a net negative charge and high encapsulation as shown above (250 μg/mL OVA in 25 mM HEPES pH 11-11.3 and 0.125-0.5 AlCl₃.6H₂O), we screened linear or branched polyethylenimine (PEI) and chitosan of various molecular weights and their ability to coat negatively charged alum complexes. We will use a three-inlet FNC device with the OVA and AlCl₃.6H₂O solutions in two separate inlets and either the LPEI or chitosan at concentrations of 10 μg/mL-10 mg/mL. The nanoparticle size, PDI, zeta potential, and encapsulation efficiency will be measured after modulating the flow rate between 0.25 mL/min-35 mL/min controlled by a syringe pump (New Era Pump Systems, Inc.). Subsequently, these polycationic nanoparticles will then be used to repeat the same studies done using HA-SH coated Alum:OVA NPs including, but not limited to: dendritic cell stimulation, T-cell stimulation and expansion, antigen-specific antibody titer and antibody isotyping after vaccination, and use of this platform as a prophylactic/therapeutic vaccines against cancer.

4.2 Stimulation and Activation of Bone Marrow derived Dendritic Cells (BMDCs) and Cytokine Secretion after Treatment by Polycationic Coated, HA-SH coated, or CpGc Coated Alum:OVA NPs.

Murine BMDCs will be isolated using widely accepted GM-CSF differentiation protocol published by Lutz et al., J Immunol Methods (1999). Day 8-10 BMDCs will be treated using varying concentrations of OVA formulations (polycationic coated Alum:OVA; HA-SH coated Alum:OVA; CpG-coated Alum:OVA, Alum:OVA complex without coating) for 12 h or 24 h in non-tissue culture treated 24-well flat-bottom or 96-well round bottom plates. Supernatants will be removed and cytokine secretion measured by Ready-Set-Go ELISA kits as per the manufacturer's instructions. To assess BMDC activation, maturation, and antigenic presentation, BMDCs will seeded in non-tissue culture treated 96-well round bottom plates and treated with varying OVA formulations at different concentrations. At 6, 12, 24, or 48 h, BMDCs will be isolated and stained for CD40, CD80, CD86, MHC class I, and MHC class II and assessed using flow cytometry (BD FACSCanto).

### 4.3 In Vitro DC and T-Cell Co-Cultures

Murine BMDCs from C57BL6/J mice at Day 8-10 will be treated using varying concentrations of OVA formulations for 12 h or 24 h in 96-well round bottom plates. OT-I and OT-II T-cells specific for OVA presented on MHC class I and MHC Class II, respectively, will be isolated from OT-I and OT-II mouse spleens (or lymph node) by digesting the tissue and collecting the T-cells using CD8 and CD4 T-cell

Isolation Kits (Miltenyi Biotec) according to the manufacturer's instructions. The OT-I or OT-II T-cells will be labeled using CellTrace™ carboxyfluorescein succinimidyl ester (CFSE) dye (Thermofisher) according to manufacturer's instructions. The OT-I or OT-II cells will then be added to already treated BMDC culture at various ratios (5:1-20:1 DC:T cells) and incubated for 3 days. Supernatant will then be harvested for cytokine analysis by ELISA. Cells will then be restimulated with Ionomycin (1 ug/mL) and phorbol 12-myristate 13-acetate (PMA, 50 ng/mL) for 2 h at 37 C. Cytokine secretion will then be stopped using Brefeldin A for 2 h. Cells will then be collected and stained for flow cytometry to assess T-cell activation, phenotype polarization, and proliferation.

4.4 Tumor-Specific Response after Prophylactic or Therapeutic Vaccination

The ability of Alum:OVA complexes with a variety of polyanionic and polycationic coatings will be assessed within the context of an anti-tumor immune response. First, we will vaccinate 6-8 week old C56BL6 mice with the nanoparticles (Days −14, −7) as a prophylactic treatment before s.c. inoculation with B16-OVA (Day 0). Similarly, we will assess these NPs as therapeutic vaccines by first inoculating 6-8 week old C57BL6 mice with B16-OVA (Day 0) s.c. followed by a prime (Day 7) and boost (Day 14) dose of the nanoparticle vaccine. We will adjust the OVA dose for each formulation to maximize the immune response. Mice will then be followed for 30-60 days depending on the tumor burden and mouse distress. Tumor burden will be measured daily using digital calipers.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.

Jiang, H. et al., Adv. Sci. 5, 1700322 (2018).

Wang, T. et al., ACS Appl. Mater. Interfaces 7, 6391-6396 (2015).

Wilson, N. S. et al., Blood 102, 2187-2194 (2003).

Howard, G. P. et al., Nano Res. 12, 837-844 (2019).

Shirodkar, S. et al., Pharmaceutical Research, 7, 1282-1288 (1990).

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A nanoparticle comprising an alum core and a coating, wherein the nanoparticle has a number average size between about 20 nm and about 300 nm and a polydispersity index between about 0.1 to about 0.3.

2. The nanoparticle of claim 1, wherein the alum core comprises an aluminum compound selected from the group consisting of aluminum hydroxide, aluminum phosphate, aluminum chloride, amorphous aluminum hydroxyphosphatesulfate (AAHS), potassium aluminum sulfate, and combinations thereof.

3. The nanoparticle of claim 1, wherein the surface coating is selected from the group consisting of one or more anionic polysaccharides, one or more nucleic acids, one or more cationic polymers, and one or more anionic polymers.

4. The nanoparticle of claim 3, wherein the one or more anionic polysaccharides is selected from the group consisting of hyaluronic acid, heparin sulfate, chondroitin sulfate, and dextran sulfate.

5. The nanoparticle of claim 3, wherein the one or more cationic polymers are selected from the group consisting of linear or branched polyethylenimine, poly(L-lysine), poly(β-amino esters), protamine, chitosan, and combinations thereof.

6. The nanoparticle of claim 3, wherein the one or more anionic polymers comprise cytosine phosphoguanosine (CpG) oligodeoxynucleotide. 7 The nanoparticle of claim 1, wherein the surface coating is crosslinked.

8. The nanoparticle of claim 4, wherein the crosslinked surface coating comprises thiolated hyaluronic acid or thiolated dextran sulfate.

9. The nanoparticle of claim 1, further comprising a protein or peptide antigen entrapped within the coating.

10. The nanoparticle of claim 1, further comprising a protein or peptide antigen conjugated to a surface of the coating.

11. The nanoparticle of claim 1, wherein the nanoparticle has a number average size between about 20 nm and about 200 nm.

12. A vaccine adjuvant comprising a nanoparticle of claim 1.

13. A vaccine comprising a nanoparticle of any of claims 1-12 or a vaccine adjuvant of claim 12.

14. The vaccine of claim 13, further comprising one or more of a vaccine selected from the group consisting of Anthrax, DT, DTaP (Daptacel), DTaP (Infanrix), DTaP-IPV (Kinrix), DTaP-IPV (Quadracel), DTaP-HepB-IPV (Pediarix), DTaP-IPV/Hib (Pentacel), HepA (Havrix), HepA (Vaqta), HepB (Engerix-B), HepB (Recombivax), HepA/HepB (Twinrix), HIB (PedvaxHIB), HPV (Gardasil9), Japanese encephalitis (Ixiaro), MenB (Bexsero,Trumenba), Pneumococcal (Prevnar13), Td (Tenivac), Td (MassBiologics), Tdap (Adacel), Tdap (Boostrix), and malaria (RTS.S (Mosquirix)).

15. The vaccine of claim 13, wherein the vaccine is a cancer vaccine.

16. The vaccine of claim 15, wherein the cancer vaccine is selected from the group consisting of BiovaxID (follicular lymphoma, a type of non-Hodgkin's lymphoma), sipuleucel-T (prostate cancer), oncophage (kidney cancer), and talimogene laherparepvec (melanoma), or a patient-derived neoantigen.

17. A method for treating a disease or condition, the method comprising administering a nanoparticle of any of claims 1-11 to a subject in need of treatment thereof.

18. The method of claim 17, wherein the treating is prophylactic.

19. The method of claim 17, wherein the treating is therapeutic.

20. The method of claim 17, wherein the nanoparticle drains to one or more lymph nodes.

21. The method of claim 17, wherein the nanoparticle induces a Th1 anti-tumor response.

22. The method of any of claims 17-21, wherein the administering of the nanoparticle is selected from the group consisting of intradermal (i.d.), subcutaneous (s.c.), and intramuscular (i.m.).

23. Use of a vaccine of any of claims 13-16 for treating or preventing an infectious disease, a cancer, and/or one or more other targets requiring cellular immunity for immunological protection.

24. The use of claim 23, wherein the use is prophylactic or therapeutic.

25. A method for preparing an alum nanoparticle, the method comprising admixing alum with a protein in a flash nanocomplexation apparatus.

26. The method of claim 25, further comprising admixing a surface coating with the alum and protein in a flash nanocomplexation apparatus.

27. The method of claim 25 or claim 26, comprising a one-step flash nanocomplexation process or a two-step flash nanocomplexation process.
